(19) United States
(12) Patent Application Publication
(10) Pub. No.: US 2014/0296733 A1
(43) Pub. Date: Oct. 2, 2014

(54) INTER-RATER AND INTRA-RATER RELIABILITY OF PHYSIOLOGICAL SCAN INTERPRETATION

(71) Applicant: BIO-SIGNAL GROUP CORP., New York, NY (US)

(72) Inventors: Ahmet Omurtag, New York, NY (US); Samah G. Abdel Baki, Brooklyn, NY (US); James G. Donnett, St. Albans (GB); Andre A. Fenton, New York, NY (US)

(21) Appl. No.: 14/355,819

(22) PCT Filed: Oct. 26, 2012
(86) PCT No.: PCT/US2012/062214

§ 371 Date: May 1, 2014

Related U.S. Application Data
(60) Provisional application No. 61/554,743, filed on Nov. 2, 2011.

(51) Int. Cl. A61B 5/00; A61B 5/04; A61B 5/0476

(52) U.S. Cl. CPC A61B 5/4094; A61B 5/0476; A61B 5/0006; A61B 5/04012; A61B 5/7475
USPC 600/544

(57) ABSTRACT

Inter-rater and intra-rater reliability of physiological scan interpretation is herein described. A plurality of physiological scan interpretations may be collected for a physiological scan. A physiological scan interpretation reliability score may be calculated for the physiological scan based on the plurality of physiological scan interpretations. When the reliability score falls below a predetermined threshold, an additional physiological scan interpretation may be collected for the physiological scan. The reliability score may be recalculated using the additional physiological scan interpretation.

### CLAIM OF PRIORITY

This patent application claims the benefit of priority, under 35 U.S.C. §119(e), to U.S. Provisional Patent Application Ser. No. 61/554,743, titled “EEG Interpretation Systems and Methods,” filed Nov. 2, 2011, which is incorporated by reference herein in its entirety.

### STATEMENT REGARDING FEDERALLY SPONSORED RESEARCH OR DEVELOPMENT

This invention was made with government support under award number 1 RC3 NS070658-01 awarded by the U.S. Department of Health and Human Services (HHS). The government has certain rights in this invention.

### BACKGROUND

A physiological scan (e.g., recordings, observations, signals, images, etc.), such as an electroencephalogram (EEG), a magnetoencephalogram (MEG), an electrocardiogram (EKG), a radiogram (including x-ray scans), a magnetic resonance imaging (MRI) scan, a tomogram (including CT or CAT scans), an ultrasonogram, an echocardiogram, or an elastogram, can be an important diagnostic tool for medical professionals, however, interpretations of physiological scans may be subjective with respect to a rater (e.g., reviewer, interpreter, observer, etc.). This subjectivity can result in a variety of diagnoses for a given physiological scan. For example, there may be variable interpretations for the same physiological scan by the same reader at different times resulting in intra-rater variability for the interpretations. There may also be variable interpretations of the same physiological scan by different readers resulting in inter-rater variability for the interpretations.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of system for physiological scan interpretation, according to an embodiment.

FIG. 2 illustrates an example of a system component for physiological scan interpretation, according to an embodiment.

FIG. 3 illustrates and example of a graphical user interface for physiological scan interpretation, according to an embodiment.

FIG. 4 illustrates an example of a method for physiological scan interpretation, according to an embodiment.

FIG. 5 is a block diagram illustrating an example of a machine upon which one or more embodiments may be implemented.

### DETAILED DESCRIPTION

These difficulties in interpreting physiological scans may impede diagnosis and treatment of a subject (e.g., patient). Availability of a skilled reader and timely physiological scan interpretation of a subject's physiological scan data may also be difficult to obtain. Although the following discussion generally discusses a solution to the difficulty of interpreting physiological scans as an illustrative example, the described systems and techniques are applicable to a variety of physiological scans. Existing efforts to monitor interpretations of physiological scan recordings from human subjects have displayed inter-rater or intra-rater variability in the physiological scan interpretations. The inter-rater and intra-rater reliability of physiological scan interpretation may have significant clinical implication because there may be no gold standard from which of the interpretations is correct. Although producing a standardized physiological scan (e.g., recording of physiological scan data) may be greatly streamlined by using appropriate digital equipment, electrode application guidelines when collecting physiological scan data, or standardized terminology for physiological scan interpretations, variability in the subjective interpretations of the scans. Even highly trained, experienced, and board certified clinical neurophysiologists are often unable to achieve agreement when they read the same physiological scan or when the same rater reads a particular physiological scan at different times.

Variable interpretations of physiological scans can impair correct diagnosis and thus treatment. For example, brain abnormalities may have severe or life threatening consequences if not diagnosed quickly. Nonconvulsive status epilepticus (NCSE) is a condition in which a patient may repeatedly have seizures without complete recovery while showing little or no clinical symptomatology. In these and other cases, an EEG scan may be the only definitive diagnostic tool. Variable interpretations of the EEG scan, or a misdiagnosis by a patient care specialist can prevent proper action from being taken to help the subject. Therefore a high degree of inter-rater and intra-rater variability of EEG interpretation may be a major impediment to reliable or effective clinical management of the subject.

Numerous studies to date have measured inter-rater variability. One study approach found slight to moderate agreement among five board certified EEG readers in reading EEGs that are twenty minutes or longer from critically ill adults. Intra-observer agreement ranged from slight to substantial depending on the category of EEG findings being evaluated. In this and other studies, rater reliability may be measured using the kappa statistic (Cohen or Fleiss) which may provide values that may be associated with degrees of agreement, ranging from slight to nearly perfect.

Another study approach evaluated inter-rater reliability between three pediatric neurophysiologists reading EEGs from critically ill children. Agreement was moderate for the presence or absence of seizure, substantial for burst suppression and overall rating, fair for interictal epileptiform discharge, and slight for focal slowing.

Abdel Baki et al. (2011) had six board-certified experts read EEGs from 200 patients one year or older and provide assessments for seven categories of EEG features. (Abdel Baki S G, Chari G, Arnedo V, Koziorynska E, Lushbough C A, Maus D, McSween T, Mortati K A, Omurtag A, Reznikov A, Weedon J, Grant A C. Inter-rater Reliability (IRR) of EEG Interpretation: A Large Single-Center Study. 65th Ann. Meeting of the American Epilepsy Soc., 2011, Baltimore Md.) Inter-rater agreement ranged from slight to moderate depending on the category. For example agreement was slight for the presence or absence of seizure or status epilepticus. The aggregated agreement over all 7 categories was in the moderate range. Abdel Baki et al. (2012) found that the average intra-rater reliability was moderate for the same six raters when they blindly re-read 100 EEGs that they had previously read approximately three months earlier. (Abdel Baki S G, Omurtag A, Weedon J, Lushbough C A, Chari G, Koziorynska E, Maus D, McSween T, Mortati K A, Reznikov A, Arnedo V, Grant A C. Intra-rater reliability of EEG interpretation: a large single-center study. 10th European Congress on Epileptology, 2012, London.)

The variability in physiological scan interpretations can reduce the diagnostic efficacy of these physiological scans. To improve diagnostic value of physiological scans, physiological scan interpretations should reliably produce substantially similar results under similar circumstances. If physiological scan interpretations are not reliable it may be difficult to draw a conclusion from the data. Furthermore, if a physiological scan is highly reliable, it may in general be easier to systematically increase its diagnostic usefulness, such as by using evidence-based methods to develop the previously discussed “gold standard.” If the reliability (e.g., repeatability) of physiological scan interpretation is improved, the medical value of physiological scans, such as for diagnosis or prognosis, can also be improved. The diagnostic utility of physiological scans would also be more amenable to further improvement.

This document describes, among other things, examples of physiological scan interpretation systems or methods. These examples may facilitate one or more of the local or remote storing, local or remote viewing, or local or remote interpretation of physiological scans. These examples may facilitate local or remote calculation or presentation of one or more derived measures (e.g., reliability scores) from physiological scan data (or derived from the interpretation of such physiological scan data). Physiological scan interpretation systems or methods may monitor, mitigate, compensate, or use the inter-rater or intra-rater reliability score component of physiological scan interpretations or one or more measures derived therefrom. The described systems or methods may include one or more features to help monitor, improve, or manage the inter-rater or intra-rater reliability of a team of raters, such as without directly modifying the physiological scan interpretation or its use in patient care.

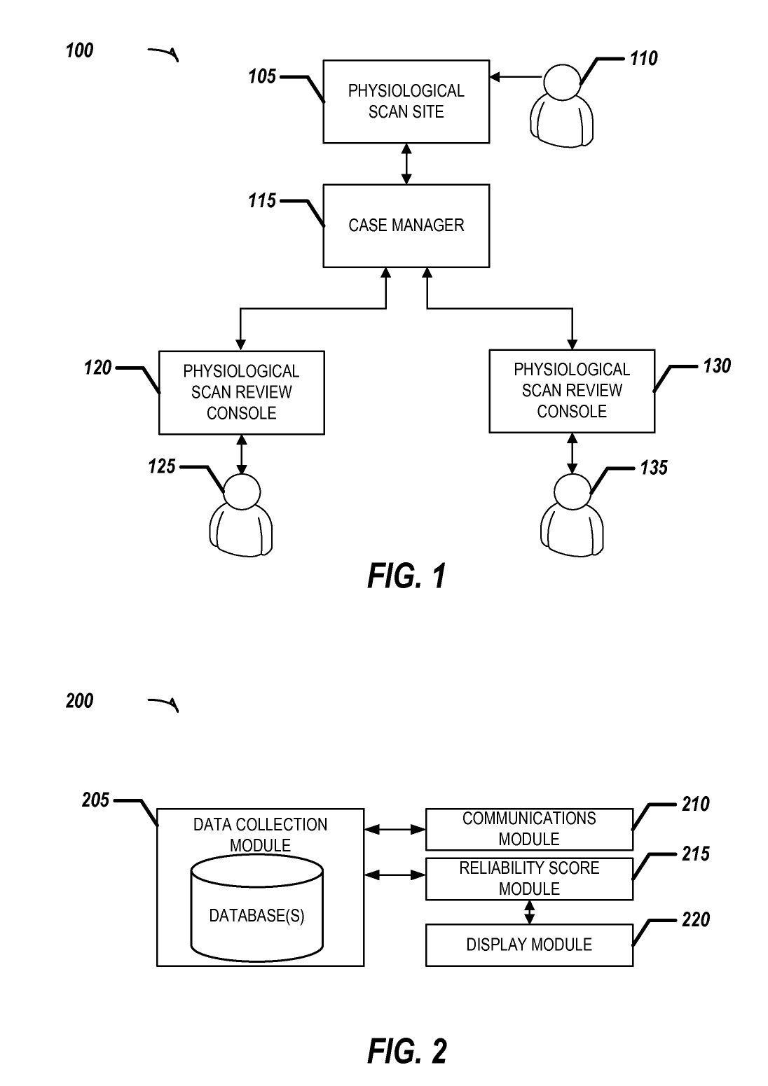

FIG. 1 illustrates an example of a system 100 for physiological scan interpretation. The system 100 may include a physiological scan site 105, a case manager 115, and one or more physiological scan review consoles 120 and 130.

The physiological scan site 105 may be arranged (e.g., configured) to produce a physiological scan of a patient 105. For example, the physiological scan site 105 can record or transmit a physiological scan. In an example, the physiological scan site 105 may be arranged to include or be used with any type of physiological scan recording system or physiological scan data storage system. In an example, the physiological scan site 105 may collect EEG data using a commercial microEEG system. In an example, the physiological scan site 105 may include using a physiological scan of any recording duration (e.g., of any amount of time). In an example, the physiological scan may be obtained at any location communicatively coupled to the physiological scan site 105, such as in any department of a hospital, doctor's office, at home, in the field, or any other location. In an example, the physiological scan site 105 may be used by patient care specialists to collect physiological scan data or to receive physiological scan interpretations. In an example, the physiological scan site 105 may stream the physiological scan data to the case manager 115. In an example, the physiological scan site 105 may transfer all physiological scan data upon completion of a physiological scan, such as when EEG recording had ceased. In an example, the physiological scan site 105 may transfer a portion of the physiological scan data at a predefined interval to the case manager 115.

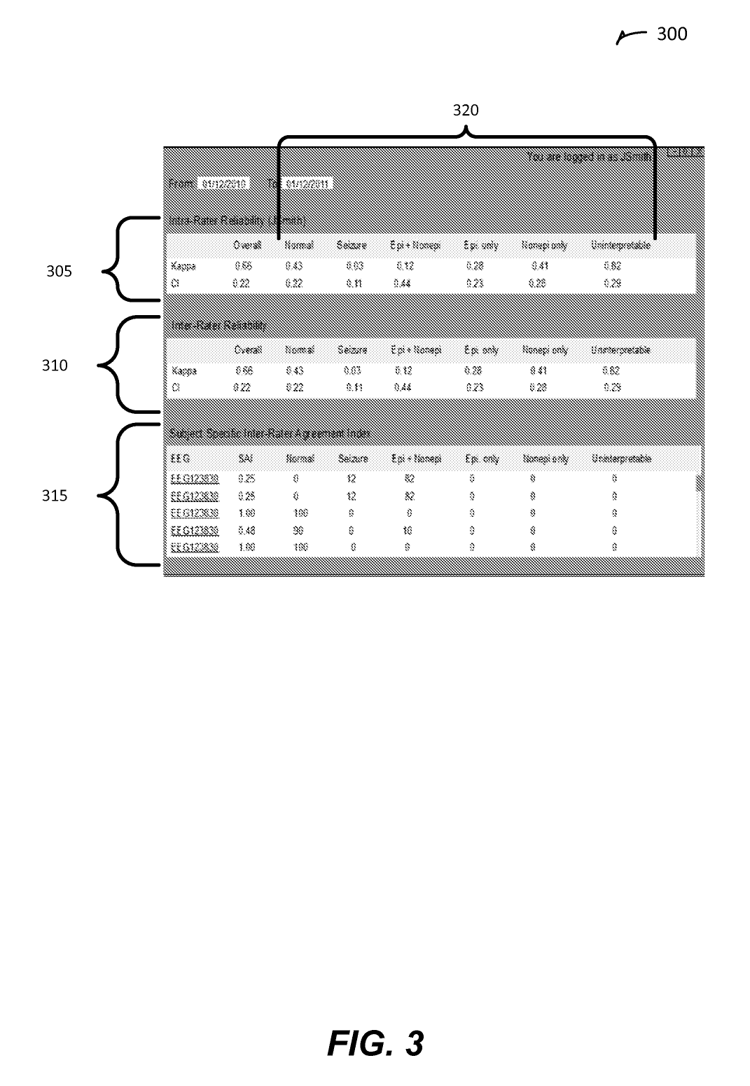

The physiological scan review console 120 may be arranged to enable physiological scan raters 125 to provide physiological scan interpretations. The physiological scan review console 120 may be arranged to accept an interpretation of a physiological scan from a physiological scan rater 125. For example, a physiological scan rater 125 may receive a physiological scan on his physiological scan review console 120 and be presented with a user interface to enter his interpretation for the physiological scan. In an example, the physiological scan rater 125 may analyze the physiological scan data, complete an interpretation form, and transfer the physiological scan interpretation to the case manager 115. In an example, physiological scan interpretations may be used by patient care specialists who receive the physiological scan interpretations submitted to the case manager 115 to inform therapeutic decisions. A graphical user interface (GUI) may be arranged to enable one or more of the discussed structures. An example embodiment of such a GUI is described below with respect to FIG. 3.

In an example, multiple physiological scan review consoles 120 and 135 may be arranged to be co-located or may be distributed across any combination of multiple centers or institutions or geographic locations. In an example, multiple physiological scan review consoles 120 and 130 may be used to collect interpretations from a plurality of physiological scan raters 125 and 135. For example, physiological scan raters 125 and 135 may include diagnostic specialists who may belong to one or more health centers. In an example, physiological scans interpreted by physiological scan raters 125 and 135 may have been recorded from one or more patients at physiological scan site 105.

In an example, the physiological scan rater 125 may be an automated (e.g., computer-implemented or device-implemented) rater module within the physiological scan review console 120. In an example, the automated rater module may be arranged to interpret the patterns in human physiological scans in order to provide an interpretation of the physiological scan to determine an underlying etiology (e.g., diagnosis). In an example, the physiological scan review console 120 may be arranged to include one or more automated physiological scan interpretation or abnormality detection modules. For example, the detection module may produce (e.g., display) an abnormality score for the whole physiological scan. In an example, the detection module may be arranged to flag one or more specific events or ranges in the physiological scan, and may be arranged to display the event or range before, during, or after the human physiological scan rater 120 completes the physiological scan interpretation.

The case manager 115 may be arranged to coordinate the collection, review, or reporting of physiological scan data and interpretations. In an example, the case manager 115 may be arranged to select a physiological scan review console 120, and transfer the recorded physiological scan data to the physiological scan review console 120. In an example, the case manager 115 may be arranged to send the physiological scan interpretation to the physiological scan site 105. In an example, the physiological scan site 105 may include a terminal from which the physiological scan result can be displayed. In an example, the case manager 115 may be arranged to send the age and medications of the patient to the physiological scan review console 120, for example, to aid the rater 125 in providing the physiological scan interpretation.

In an example, the case manager 115 may be arranged to recurrently or periodically send a modified (e.g., de-identified) physiological scan (e.g., in which individually identifiable patient characteristics are removed) to one or more physiological scan review consoles 120 and 130. For example, modified physiological scan may be used for blind scoring. In an example, the modified physiological scan may include one or more modifications to the original physiological scan to make it more difficult for a rater to recognize the data without impairing diagnostic characteristics of the data. In an example, such modification can include one or more of using (e.g., randomly alternating between) different colors (e.g., foreground and background colors), different clipping windows, re-scaling (e.g., resizing), different file names, etc. In an example, the case manager 115 may be arranged to collect (e.g., receive and store) the modified physiological scans and their interpretations over a predetermined set of categories. In an example, the case manager 115 may be configured to send a previously interpreted physiological scan to a physiological scan review console 120 for an additional interpretation. For example, the previously interpreted physiological scan may be sent as a modified physiological scan after a predetermined period of time.

In an example, the case manager 115 may be arranged to include one or more automated physiological scan interpretation or abnormality detection modules. For example, these modules may be arranged to produce an abnormality score for the whole physiological scan, or may flag one or more specific events or ranges of the physiological scan. In an example, these modules may be arranged produce a score or flag arranged to alert one or more of the raters 125 or 135 before, during, or after the physiological scan interpretation is complete. An example embodiment of the case manager 115 is discussed below with respect to FIG. 2.

FIG. 2 illustrates an example of a system 200 implementing inter-rater and intra-rater reliability score monitoring of physiological scan interpretation. The system 200 may include a data collection module 205, a communications module 210, a reliability score module 215, and a display module 220.

The communications module 210 may be arranged to send and receive data on behalf of one or more of the data collection module 205, the reliability score module 215, or the display module 220. For example, the communications module may be arranged to receive a physiological scan interpretation request from the data collection module 205 and use a cellular network to communicate the request to a, for example, physiological scan review console 130.

The display module 220 may be arranged to display one or more warning messages to a rater 125 during or after the interpretation of patient physiological scans. In an example, the display module 220 may be arranged to display a warning message when the rater's physiological scan interpretation result is one of controversial pattern set. Members of the controversial pattern set include interpretations that have been previously determined to lead to low inter-rater agreement or low intra-rater agreement. In an example, the display module 220 may produce the displayed information directly, such as one a computer screen. In an example, the display module 220 may be arranged to produce a graphical object that can be rendered by another device. For example, the display module 220 may produce a Hypertext Markup Language (HTML) document that can be rendered on a web browser. In an example, the display module 220 can be arranged to serve data that can be used by another device to render a display. For example, the display module 220 may be a web server, or simple object access protocol (SOAP) service, among others. An example embodiment of a GUI that the display module may produce is described below with respect to FIG. 3.

The data collection module 205 may be arranged to collect physiological scans or physiological scan interpretations. In an example, the data collection module 205 may be arranged to store collected physiological scans or physiological scan interpretations. In an example, the data collection module 205 may include a database arranged to store physiological scan data. In an example, the data collection module 205 may direct the communications module 210 to send additional physiological scan data to the data collection module 205.

The reliability score module 215 may be arranged to retrieve physiological scan data from the data collection module 205. In an example, the reliability score module 215 may retrieve a subset of the physiological scan data from the data collection module 205, and may compute a reliability score for that subset of physiological scan data. In an example, the reliability score can include one or more components, such as an intra-rater, inter-rater, or diagnosis component.

In an example, the reliability score module 215 may be arranged to monitor a correlation between a rater's intra-rater reliability and one or any combination of that person's individual characteristics. For example, these characteristics may include one or more of: length of training; board membership; activity level, e.g., number of physiological scans read per week or other unit time; experience, e.g., total number of physiological scans read to date; or specialty (e.g., pediatric vs. adult electroencephalography).

In an example, the reliability score module 215 may be arranged to monitor a correlation between physiological scan specific (e.g., for a particular physiological scan) inter-rater agreement. In an example, the reliability score module 215 may be arranged to monitor one or more individual physiological scan characteristics. For example, these characteristics may include one or more of: the category into which the particular physiological scan was classified; patient age; patient medications; or patient outcome.

In an example, the reliability score module 215 may be arranged to allow the physiological scan rater 125 to modify the day-to-day practice of physiological scan interpretation. For example, the reliability score module may be arranged to allow the physiological scan rater 125 to integrate or otherwise use specific inter-rater or intra-rater reliability information into the physiological scan interpretation procedure. In an example, the reliability score module 215 may be arranged to provide a mechanism to seek, promote, or obtain consensus among raters, such as a voting system to determine a diagnosis before a patient with intractable epilepsy is considered for surgery.

In an example, the reliability score module 215 may be arranged to cause the display module 220 to make the physiological scan interpretation available for viewing or other access, such as by the patient care specialist. For example, the reliability score module 215 may be arranged to cause the display module 220 to make multiple physiological scan interpretations or a composite interpretation available to the patient care specialist. In an example, the reliability score module 215 may be arranged to cause the display module 220 to make the level of inter-rater or intra-rater agreement associated with the physiological scan interpretation or the physiological scan rater available to the patient care specialist.

In an example, the reliability score module 215 may be arranged to monitor the intra-rater reliability of one or more individual members of a team or other group of raters. For example, the team's current degree of intra-rater reliability (e.g., as measured by Cohen's kappa or confidence interval (CI)) and its history may be calculated, stored, displayed, reported, or otherwise used. In an example, the team's current degree of intra-rater reliability may be used to adjust compensation, awards, or any other metric that may incentivize reducing the intra-rater variability.

In an example, the reliability score module 215 may be arranged to monitor the inter-rater reliability of a team or other group of physiological scan readers. For example, the current degree of inter-rater reliability for the team or group (e.g., as measured by Fleiss kappa or confidence interval (CI)) or its history may be calculated, stored, displayed, reported, or otherwise used. In an example, the current degree of inter-rater reliability may be used to adjust compensation or any other metric that may incentivize reducing the inter-rater variability. In an example, the inter-rater reliability score may also be used to monitor the physiological scan specific inter-rater agreement of a team or other group of physiological scan readers. In an example, the team's degree of agreement of a specific physiological scan may be calculated by, for example, a modified Fleiss kappa, which may be referred to as the Specific Agreement Index (SAI):

$$\text{?} = \frac{\text{?}}{\text{?}} \\ \text{?} \text{indicates text missing or illegible when filed} \qquad Eq . 1$$

where Pᵢ, the extent to which raters agree for the i-th physiological scan (which represent how many rater-rater pairs are in agreement, relative to the number of all possible rater-rater pairs), may be given by:

$$\text{?} = \text{?} \frac{\text{?}}{\text{?}} \\ \text{?} \text{indicates text missing or illegible when filed} \qquad Eq . 2$$

where nᵢⱼ may represent the rater who classified the i-th physiological scan into category j and n may represent the total number of raters in the team. For example, category j may represent a diagnosis of one or more of seizure, status epilepticus, normal, burst suppression, slowing, triphasic waves, technically limited, technically inadequate, epileptiform only, or a combination of epileptiform and nonepileptiform. For example, Pₑ may represent the chance level of agreement, and may differ from ordinary Fleiss kappa in that Pₑ averages Pᵢ over all physiological scans before calculating kappa. In an example, physiological scans with a classification into a category that was done by a large consensus will have SAI nearly equal to one, whereas low consensus physiological scans will have SAI nearly equal to zero. In an example, the SAI may be used to make the team or group aware of which physiological scans are the most controversial or the least controversial.

In an example, the reliability score module 215 may be arranged to monitor the inter-rater agreement of a team or other group of physiological scan readers. In an example, the reliability score module 215 may be used to evaluate seizure outcome, such as following epilepsy surgery, or when using more than one outcome classification. In an example, the degree of agreement among raters, or among various classification algorithms, or both, may be stored, displayed, reported, or otherwise used, for one or more individual patients following resective or disconnective epilepsy surgery.

In an example, the reliability score module 215 may be arranged to monitor the physiological scan inter-rater agreement of a team or group, such as the staff of a third or fourth level medical epilepsy center. In an example, monitoring the physiological scan inter-rater agreement may be used in selecting which patients qualify for an epilepsy surgery or other specific medical procedure.

In an example, the reliability score module 215 may be arranged to monitor the inter-rater agreement of multiple raters (e.g., raters 125 and 135) under different circumstances. For example, inter-rater agreement may be used to test one or more reduced physiological scan configurations or for developing or optimizing a reduced functional set of electrodes for an EEG scan. For example, the inter-rater agreement may provide context (e.g., how unsure the field is as to a single diagnosis) to a rater 125 to help detection of one or more physiological scan abnormalities (e.g., such as one or more seizures). In an example, inter-rater agreement may be used to attain higher correlation and consistency among any combination of human or automated raters.

In an example, the reliability score module 215 may be arranged to monitor inter-rater agreement of physiological scan readers, such as for evaluating, testing, verifying, or validating adequacy of screening capability of a full or reduced physiological scan electrode/signal montage. For example, the reliability score module 215 may validate the screening capability adequacy for a recording having a duration that is abbreviated to less than the standard thirty minute screening time, either for the use of one or more raters to detect one or more physiological scan abnormalities (such as one or more seizures). For example, such testing and development may be used to attain higher correlation and consistency among raters.

In an example, the reliability score module 215 may be arranged to monitor one or more other or additional measures of inter-rater or intra-rater variability. Examples of inter-rater or intra-rater variability measures may include: (1) one or more agreement scores among all or a selected group of readers (e.g., kappa); (2) one or more agreement scores among particular rater pairs (e.g., kappa); (3) latent class analysis such as to identify which physiological scan categories created a uniform or non-uniform pattern of association among the raters (e.g., relating the inter-rater agreement to one or more of the characteristics of the physiological scan category being assessed); or (4) one or more agreement scores such as between any combination of human or automated raters.

In an example, the reliability score module 215 may be arranged to include one or more features that may be configured to mitigate inter-rater reliability or intra-rater reliability. In an example, such features may be arranged to directly or indirectly modify the process of physiological scan interpretation and its use in patient care. For example, this may include using automated physiological scan interpretation techniques to recognize or classify a particular physiological scan as being of a type (e.g., class) that is similar to a known (e.g., template, type or class) physiological scan that is known to be particularly problematic (e.g., is associated with large variability in inter-rater or intra-rater interpretation). In an example, recognizing or classifying a physiological scan may prompt the physiological scan rater 125 to take additional or time to perform or care in performing the interpretation of that particular physiological scan.

In an example, the reliability score module 215 may include one or more automation modules that may be arranged to determine whether the physiological scan should be re-interpreted after the interpretation is completed. In an example, the automation modules may be arranged to determine whether the same rater or a different rater should re-interpret the physiological scan. In an example, the automation modules may be arranged to facilitate (e.g., rank, recommend, etc.) which raters among a plurality of raters should be used as re-rater. In an example, the reliability score module 215 may include a combination module arranged to combine multiple physiological scan interpretations for the same physiological scan into a single (e.g., composite) physiological scan interpretation. This may be accomplished by weighting, voting, or otherwise deciding what data from the plurality of interpretations will be included. In an example, the reliability score module 215 may include a choosing module arranged to choose the interpretation to be used in patient care, such as using previously-collected intra-rater reliability data.

In an example, the reliability score module 215 may be arranged to monitor the intra-rater reliability or inter-rater reliability of the team of raters, or direct the display module 220 to make available for viewing one or more measures related to their intra-rater reliability or inter-rater reliability. In an example, the reliability score module 215 may display (e.g., via the display module 220) or send (e.g., via the communications module 210) one or more warning messages to the patient care specialist or other user, such as when the physiological scan interpretation or physiological scan rater is associated with low inter-rater or intra-rater agreement, or both. In an example, an individual rater or a team that is attempting to improve its interpretations may continually (e.g., recurrently, continuously, etc.) use the information presented to them by the present systems or methods, such as over an extended period of time. In an example, to gain insight into one or more causes of variability in physiological scan interpretation, the reliability score module 215 may be arranged to allow a group of raters to choose to have (e.g., schedule) recurrent or periodic meetings, such as to review physiological scans with particularly high or low inter-rater or intra-rater variability.

In an example, the reliability score module 215 may be arranged to direct the display module 220 to display (e.g., via the display module 220) a warning message to the patient care specialist or other user when the interpretation result is at least one of a commonly overread patterns. For example, the “overreading” of benign physiological scan patterns (e.g., misclassifying a benign physiological scan pattern as symptomatic of a disease condition or other abnormality) may contribute to the misdiagnosis of epilepsy. In an example, the reliability score module 215 may be arranged to direct the display module 220 to prompt a rater to prescreen (e.g., interpret) one or more physiological scans. For example, the prescreen interpretation may be performed using a subset (e.g., reduced montage) selected from available physiological scan channels. For example, the prescreening physiological scan channel subset may be selected to carry discriminative information. For example, the prescreening physiological scan channel subset may be selected to produce high inter-rater or intra-rater agreement. In an example, prescreening results may be arranged to produce one or more abnormality scores. For example, the abnormality scores may be arranged to identify one or more specific events or physiological scan ranges. In an example, the prescreening results may be made available to one or more raters before, during, or after the physiological scan interpretation is complete. In an example, the reliability score module 215 may be arranged to direct the display module 220 to present one or more of the inter-rater or intra-rater reliability results. For example, the inter-rater or intra-rater reliability results may be displayed in a spreadsheet (e.g., table, chart, etc.).

In an example, the reliability score module 215 or display module 220 may be coupled to (e.g., as a “plug-in”) an existing computer-implemented or device-implemented environment. For example, physiological scan rater may use the existing computer-implemented or device-implemented environment to view physiological scan results.

In an example, the reliability score module 215 may be arranged to include an interface module. For example, the interface module may be arranged to create, provide, or send one or more customizable reports (e.g., custom time, custom content, etc.) to one or more users (e.g., a group leader, a single team member, multiple team members, etc.). In an example, the customizable report may be arranged to include inter-rater or intra-rater reliability for an individual rater or a group of raters.

FIG. 3 illustrates an example physiological scan interpretation graphical user interface (GUI) 300 for inter-rater and intra-rater reliability of physiological scan interpretation. The physiological scan interpretation GUI 300 may include an intra-rater reliability display 305, an inter-rater reliability display 310, or subject-specific inter-rater agreement index display 315. In an example, the physiological scan interpretation GUI 300 may include reliability scores for various physiological scan diagnoses 320. In an example, the physiological scan interpretation GUI 300 may be arranged so that rows may be sorted by clicking on the appropriate column header. In an example, the intra-rater reliability display 305 or the inter-rater reliability display 310 may be arranged to include a confidence interval, abbreviated CI. In an example, the physiological scan interpretation GUI 300 may be arranged to allow the intra-rater reliability display 305 or the inter-rater reliability display 310 to be hidden. For example, the intra-rater reliability display 305 or the inter-rater reliability display 310 may be hidden when the system is used for a discussion (e.g., presentation, meeting, etc.). In an example, the subject-specific inter-rater agreement index display 315 may be arranged to include physiological scan file names. In an example, a physiological scan file may be arranged to include patient characteristics. In an example, clicking on a physiological scan file name may launch the physiological scan in a commercial physiological scan viewer. In an example, a physiological scan file may be viewed in a commercial physiological scan viewer to allow for analysis of high- or low-consensus physiological scans.

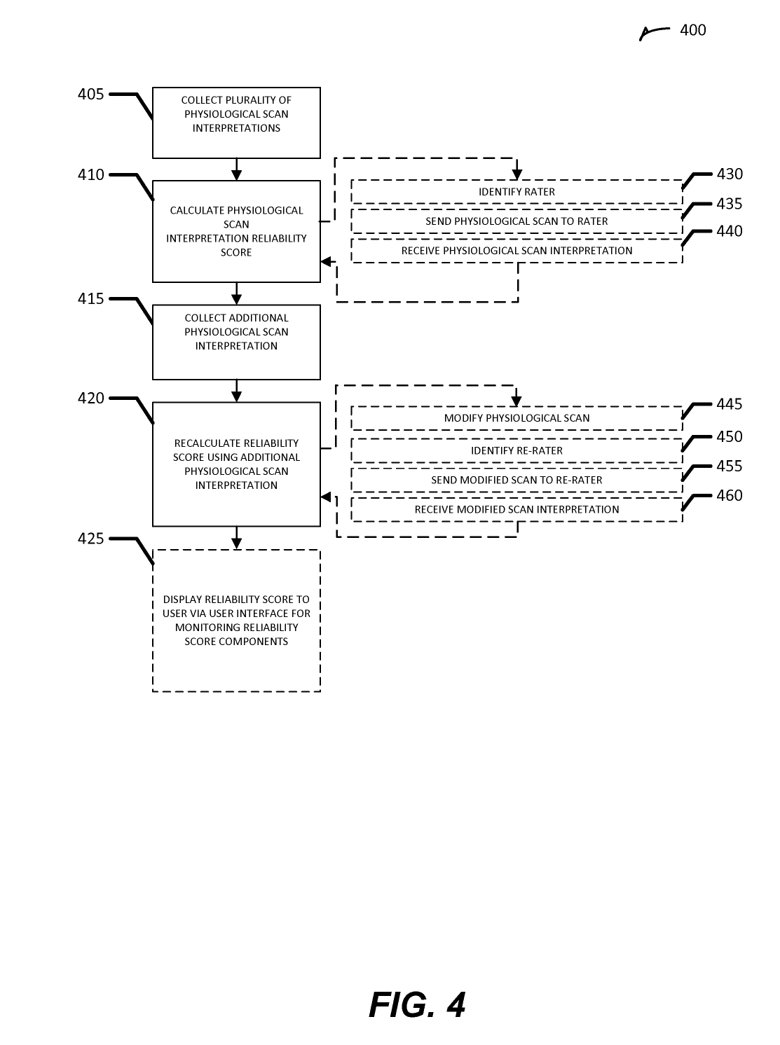

FIG. 4 illustrates an example of a method for inter-rater and intra-rater reliability of physiological scan interpretation 400. The operations of method 400 may be performed in whole or part by one or more components described above with respect to FIGS. 1-3.

At operation 405, a plurality of physiological scan interpretations are collected for a particular physiological scan. In an example, the physiological scan interpretations are collected from different raters. In an example, the physiological scan interpretations are collected from a single physiological scan rater.

At operation 410, the physiological scan interpretation reliability score is calculated. In an example, calculating a physiological scan interpretation reliability score 410 includes identifying a rater 430, sending the physiological scan to the rater 435, or receiving the physiological scan interpretation 440 from the rater.

At operation 415, an additional physiological scan interpretation is collected. In an example, the additional physiological scan interpretation is collected from a physiological scan rater that previously interpreted the physiological scan. In an example, the additional physiological scan interpretation is collected from a physiological scan rater that did not previously interpret the physiological scan. In an example, the additional physiological scan interpretation is collected when the reliability score falls below a predetermined threshold. In an example, the additional physiological scan interpretation is collected regardless of whether the reliability score falls below a predetermined threshold.

At operation 420, the reliability score using the additional physiological scan interpretation may be recalculated. In an example, recalculating the reliability score using the additional physiological scan interpretation 420 includes modifying a physiological scan 445, identifying a re-rater 450 (e.g., entity to provide the additional physiological scan interpretation), sending the modified scan to the re-rater 455, or receiving the modified scan interpretation 460 from the re-rater. In an example, modifying a physiological scan 445 includes transforming the physiological scan into a modified physiological scan by anonymizing identifiable characteristics while retaining diagnostic characteristics, the identifiable characteristics distinguishing the physiological scan from other physiological scans. In an example, modifying a physiological scan 445 includes cropping the physiological scan, scaling the physiological scan, or modifying the foreground or background colors of the physiological scan.

At operation 425, the recalculated reliability score using the additional physiological scan interpretation may be displayed. In an example, the reliability score may be displayed to a user via a user interface for monitoring reliability score components. In an example, the reliability score is displayed as described above with respect to FIG. 3.

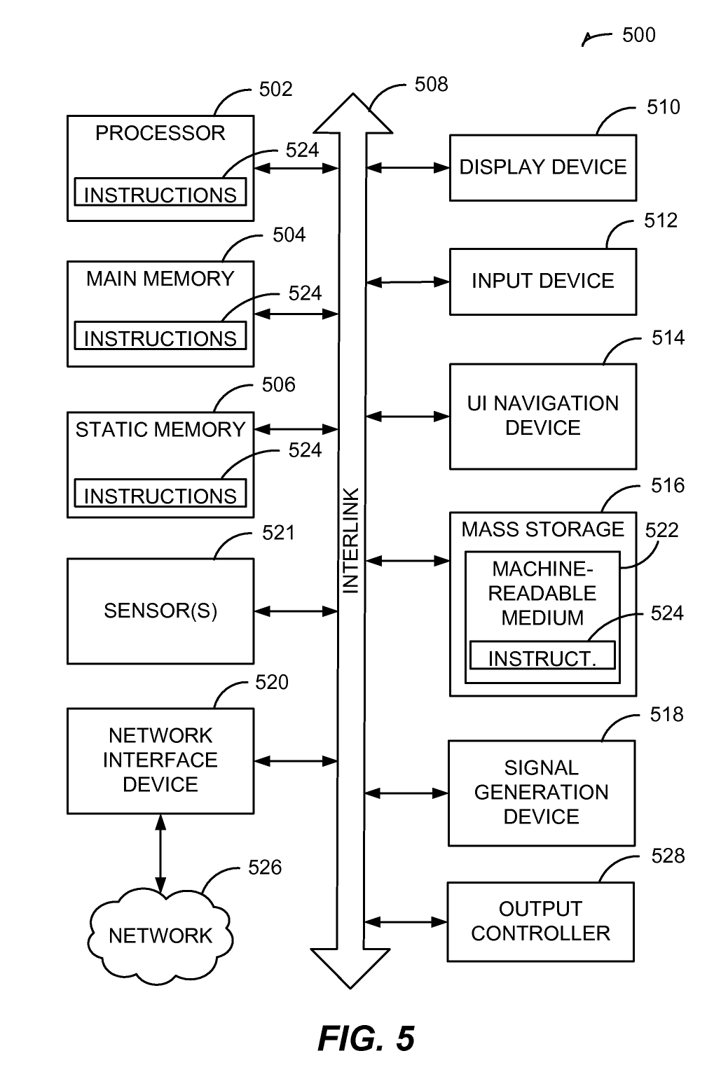

FIG. 5 illustrates a block diagram of an example machine 500 upon which any one or more of the techniques (e.g., methodologies) discussed herein may perform. In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a Personal Digital Assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Machine (e.g., computer system) 500 may include a hardware processor 502 (e.g., a central processing unit (CPU), a graphics processing unit (GPU), a hardware processor core, or any combination thereof), a main memory 504 and a static memory 506, some or all of which may communicate with each other via an interlink (e.g., bus) 508. The machine 500 may further include a display unit 510, an alphanumeric input device 512 (e.g., a keyboard), and a user interface (UI) navigation device 514 (e.g., a mouse). In an example, the display unit 510, input device 512 and UI navigation device 514 may be a touch screen display. The machine 500 may additionally include a storage device (e.g., drive unit) 516, a signal generation device 518 (e.g., a speaker), a network interface device 520, and one or more sensors 521, such as a global positioning system (GPS) sensor, compass, accelerometer, or other sensor. The machine 500 may include an output controller 528, such as a serial (e.g., universal serial bus (USB), parallel, or other wired or wireless (e.g., infrared (IR)) connection to communicate or control one or more peripheral devices (e.g., a printer, card reader, etc.).

The storage device 516 may include a machine readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 516 may constitute machine readable media.

While the machine readable medium 522 is illustrated as a single medium, the term “machine readable medium” may include a single medium or multiple media (e.g., a centralized or distributed database, and/or associated caches and servers) that arranged to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine readable medium examples may include solid-state memories and optical and magnetic media. In an example, a massed machine readable medium comprises a machine readable medium with a plurality of particles having resting mass. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, wireless data networks (e.g., Institute of Electrical and Electronics Engineers (IEEE) 802.11 family of standards known as Wi-Fi®, and IEEE 802.16 family of standards known as WiMax®), and peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

### Additional Notes & Examples

Example 1 may include subject matter (such as a method, means for performing acts, or machine readable medium including instructions that, when performed by a machine cause the machine to performs acts) comprising collecting a plurality of physiological scan interpretations for a physiological scan using a data collection module, calculating a physiological scan interpretation reliability score for the physiological scan based on the plurality of physiological scan interpretations using a reliability score module, the reliability score falling below a predetermined threshold, collecting an additional physiological scan interpretation for the physiological scan using the data collection module, and recalculating the reliability score using the additional physiological scan interpretation using the reliability score module.

In Example 2, the subject matter of Example 1 may optionally include the physiological scan being one of an electroencephalogram (EEG), a magnetoencephalogram (MEG), an electrocardiogram (EKG), a radiogram, a magnetic resonance imaging (MRI) image, a tomogram, an ultrasonogram, an echocardiogram, or an elastogram.

In Example 3, the subject matter of one or more of Examples 1-2 may optionally include at least a first physiological scan interpretation and a second physiological scan interpretation being received from different raters.

In Example 4, the subject matter of one or more of Examples 1-3 may optionally include at least a first physiological scan interpretation and a second physiological scan interpretation being received from a single rater.

In Example 5, the subject matter of one or more of Examples 1-4 may optionally include the additional physiological scan interpretation in the plurality of physiological scan interpretations including at least one diagnosis.

In Example 6, the subject matter of Example 5 may optionally include the additional physiological scan interpretation in the plurality of physiological scan interpretations including supplemental interpretation information.

In Example 7, the subject matter of one or more of Examples 5-6 may optionally include the physiological scan being an EEG, and wherein the at least one diagnosis being one of seizure, status epilepticus, normal, burst suppression, epileptiform, slowing, triphasic waves, technically limited, or technically inadequate.

In Example 8, the subject matter of one or more of Examples 1-7 may optionally include identifying a rater, sending the physiological scan to the rater, and receiving a physiological scan interpretation for the physiological scan from the rater.

In Example 9, the subject matter of one or more of Examples 1-8 may optionally include transforming the physiological scan into a modified physiological scan by anonymizing identifiable characteristics while retaining diagnostic characteristics, and the identifiable characteristics distinguishing the physiological scan from other physiological scans.

In Example 10, the subject matter of Example 9 may optionally identifying a re-rater, sending the modified physiological scan to the re-rater, and receiving the additional physiological scan interpretation for the modified physiological scan from the re-rater.

In Example 11, the subject matter of one or more of Examples 1-10 may optionally include displaying the reliability score to a user via a user interface, and the user interface being configured to permit the user to continually monitor the reliability score components of the reliability score.

In Example 12, the subject matter of Example 11 may optionally include the reliability score components including a diagnosis component respective to a diagnosis from a physiological scan interpretation from the plurality of physiological scan interpretations.

In Example 13, the subject matter of one or more of Examples 11-12 may optionally include the reliability score components including an inter-rater component, and the inter-rater component being configured to represent interpretation variability between a plurality of raters.

In Example 14, the subject matter of one or more of Examples 11-13 may optionally include the components including an intra-rater component, and the intra-rater component being configured to represent interpretation variability by a single rater.

Example 15 may include, or may optionally be combined with the subject matter of one or more of Examples 1-14 to include subject matter (such as a device, apparatus, or computing device for application independent content control) comprising a data collection module and a reliability score module. The data collection module may be configured to collect physiological scan interpretations for a physiological scan. The reliability score module may be configured to calculate a physiological scan interpretation reliability score for the physiological scan based on a plurality of physiological scan interpretations collected by the data collection module, the reliability score falling below a predetermined threshold, instruct the data collection module to collect an additional physiological scan interpretation for the physiological scan, and recalculate the reliability score using the additional physiological scan interpretation.

In Example 16, the subject matter of Example 15 may optionally include the physiological scan being one of an electroencephalogram (EEG), a magnetoencephalogram (MEG), an electrocardiogram (EKG), a radiogram, a magnetic resonance imaging (MRI) image, a tomogram, an ultrasonogram, an echocardiogram, or an elastogram.

In Example 17, the subject matter of one or more of Examples 15-16 may optionally include at least a first physiological scan interpretation and a second physiological scan interpretation being received from different raters.

In Example 18, the subject matter of one or more of Examples 15-17 may optionally include at least a first physiological scan interpretation and a second physiological scan interpretation being received from a single rater.

In Example 19, the subject matter of one or more of Examples 15-18 may optionally include each physiological scan interpretation in the plurality of physiological scan interpretations including at least one diagnosis.

In Example 20, the subject matter of Example 19 may optionally include the additional physiological scan interpretation in the plurality of physiological scan interpretations including supplemental interpretation information.

In Example 21, the subject matter of one or more of Examples 19-20 may optionally include the physiological scan being an EEG, and at least one diagnosis being one of seizure, status epilepticus, normal, burst suppression, epileptiform, slowing, triphasic waves, technically limited, or technically inadequate.

In Example 22, the subject matter of one or more of Examples 15-21 may optionally include the data collection module being configured to identify a rater, send the physiological scan to the rater, and receive a physiological scan interpretation for the physiological scan from the rater.

In Example 23, the subject matter of one or more of Examples 15-22 may optionally include the data collection module being configured to transform the physiological scan into a modified physiological scan by anonymizing identifiable characteristics while retaining diagnostic characteristics, where the identifiable characteristics distinguish the physiological scan from other physiological scans.

In Example 24, the subject matter of one or more of Examples 15-23 may optionally include the data collection module being configured to identify a re-rater, send the modified physiological scan to the re-rater, and receive the additional physiological scan interpretation for the modified physiological scan from the re-rater.

In Example 25, the subject matter of one or more of Examples 15-24 may optionally include the reliability score module being configured to display the reliability score to a user via a user interface, and the user interface being configured to permit the user to continually monitor the reliability score components of the reliability score.

In Example 26, the subject matter of Example 25 may optionally include the reliability score components including a diagnosis component respective to a diagnosis from a physiological scan interpretation from the plurality of physiological scan interpretations.

In Example 27, the subject matter of one or more of Examples 25-26 may optionally include the reliability score components including an inter-rater component configured to represent interpretation variability between a plurality of raters.

In Example 28, the subject matter of one or more of Examples 25-27 may optionally include the components including an intra-rater component configured to represent interpretation variability by a single rater.

Example 29 may include, or may optionally be combined with the subject matter of one or more of Examples 1-28 to include subject matter (such as a method, means for performing acts, or machine readable medium including instructions that, when performed by a machine cause the machine to performs acts) comprising collecting physiological scan interpretations for a physiological scan, calculating a physiological scan interpretation reliability score for the physiological scan based on a plurality of physiological scan interpretations, the reliability score falling below a predetermined threshold, collecting an additional physiological scan interpretation for the physiological scan and recalculating the reliability score using the additional physiological scan interpretation.

In Example 30, the subject matter of Example 29 may optionally include the physiological scan being one of an electroencephalogram (EEG), a magnetoencephalogram (MEG), an electrocardiogram (EKG), a radiogram, a magnetic resonance imaging (MRI) image, a tomogram, an ultrasonogram, an echocardiogram, or an elastogram.

In Example 31, the subject matter of one or more of Examples 29-30 may optionally include at least a first physiological scan interpretation and a second physiological scan interpretation being received from different raters.

In Example 32, the subject matter of one or more of Examples 29-31 may optionally include at least a first physiological scan interpretation and a second physiological scan interpretation being received from a single rater.

In Example 33, the subject matter of one or more of Examples 29-32 may optionally include each physiological scan interpretation in the plurality of physiological scan interpretations including at least one diagnosis.

In Example 34, the subject matter of Example 33 may optionally include the additional physiological scan interpretation in the plurality of physiological scan interpretations including supplemental interpretation information.

In Example 35, the subject matter of one or more of Examples 33-34 may optionally include the physiological scan being an EEG, and the at least one diagnosis being one of seizure, status epilepticus, normal, burst suppression, epileptiform, slowing, triphasic waves, technically limited, or technically inadequate.

In Example 36, the subject matter of one or more of Examples 29-35 may optionally include identifying a rater, sending the physiological scan to the rater, and receiving a physiological scan interpretation for the physiological scan from the rater.

In Example 37, the subject matter of one or more of Examples 29-36 may optionally include transforming the physiological scan into a modified physiological scan by anonymizing identifiable characteristics while retaining diagnostic characteristics, where the identifiable characteristics distinguish the physiological scan from other physiological scans.

In Example 38, the subject matter of Example 37 may optionally include identifying a re-rater, sending the modified physiological scan to the re-rater, and receiving the additional physiological scan interpretation for the modified physiological scan from the re-rater.

In Example 39, the subject matter of one or more of Examples 29-38 may optionally include displaying the reliability score to a user via a user interface, and the user interface being configured to permit the user to continually monitor the reliability score components of the reliability score.

In Example 40, the subject matter of Example 39 may optionally include the reliability score components including a diagnosis component respective to a diagnosis from a physiological scan interpretation from the plurality of physiological scan interpretations.

In Example 41, the subject matter of one or more of Examples 39-40 may optionally include the reliability score components including an inter-rater component configured to represent interpretation variability between a plurality of raters.

In Example 42, the subject matter of one or more of Examples 39-41 may optionally include an intra-rater component configured to represent interpretation variability by a single rater.

The above detailed description includes references to the accompanying drawings, which form a part of the detailed description. The drawings show, by way of illustration, specific embodiments in that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “second,” and “third,” and so forth are used merely as labels, and are not intended to impose numerical requirements on their objects.

The above description is intended to be illustrative and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure, for example, to comply with 37 C.F.R. §1.72(b) in the United States of America. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. An automated method of determining intra-rater reliability and inter-rater reliability for an electroencephalogram (EEG), the method comprising:
collecting, using a data collection module, a plurality of physiological scan interpretations for the EEG;
transmitting at least some of the plurality of physiological scan interpretations to an EEG interpretation reliability score module;
automatically calculating, using the EEG interpretation reliability score module, an intra-rater reliability score and an inter-rater reliability score for the physiological scan based on the plurality of physiological scan interpretations;
over an extended period of time:
collecting, using the data collection module, additional physiological scan interpretations for the physiological scan;
transmitting the additional physiological scan interpretations to the reliability score module;
automatically recurrently recalculating, using the reliability score module, the intra-reliability reliability score and the inter-rater reliability score using the additional physiological scan interpretations; and
providing, to a user interface, the recalculated intra-rater reliability score and the recalculated inter-rater reliability score to provide on-going monitoring.

2. (canceled)

3. The method of claim 1, wherein at least a first physiological scan interpretation and a second physiological scan interpretation are received from different raters.

4. The method of claim 1, wherein at least a first physiological scan interpretation and a second physiological scan interpretation are received from a single rater.

5. The method of claim 1, wherein the additional physiological scan interpretation in the plurality of physiological scan interpretations includes at least one diagnosis.

6. The method of claim 5, wherein the additional physiological scan interpretation in the plurality of physiological scan interpretations includes supplemental interpretation information.

7. The method of claim 5, wherein the at least one diagnosis is one of seizure, status epilepticus, normal, burst suppression, epileptiform, slowing, triphasic waves, technically limited, or technically inadequate.

8. The method of claim 1, wherein collecting the plurality of physiological scan interpretations includes:
identifying a rater;
sending the physiological scan to the rater; and
receiving a physiological scan interpretation for the physiological scan from the rater.

9. The method of claim 1, wherein collecting the additional physiological scan interpretations includes transforming the physiological scan into a modified physiological scan by anonymizing identifiable characteristics while retaining diagnostic characteristics, the identifiable characteristics distinguishing the physiological scan from other physiological scans.

10. The method of claim 9, wherein collecting the additional physiological scan interpretations includes:
identifying a re-rater;
sending the modified physiological scan to the re-rater; and
receiving the additional physiological scan interpretations for the modified physiological scan from the re-rater.

11. (canceled)

12. The method of claim 1, wherein the reliability score components include a diagnosis component respective to a diagnosis from a physiological scan interpretation from the plurality of physiological scan interpretations.

13. The method of claim 1, wherein the inter-rater component is configured to represent interpretation variability between a plurality of raters.

14. The method of claim 1, wherein the intra-rater component is configured to represent interpretation variability by a single rater.

15. A system for determining intra-rater reliability and inter-rater reliability for an electroencephalogram (EEG), the system comprising:
a data collection module configured to:
collect physiological scan interpretations for a physiological scan for the EEG; and
an EEG interpretation reliability score module configured to:
automatically calculate a an intra-rater reliability score and an inter-rater reliability score for the physiological scan based on a plurality of physiological scan interpretations collected by the data collection module;
over an extended period of time, the EEG interpretation reliability score module further configured to:
instruct the data collection module to collect additional physiological scan interpretations for the physiological scan; and
recalculate the reliability score using the additional physiological scan interpretations; and
a user interface configured to display the recalculated intra-rater reliability score and the recalculated inter-rater reliability score to provide on-going monitoring.

16-42. (canceled)

43. An automated method of determining intra-rater reliability and inter-rater reliability for an electroencephalogram (EEG), the method comprising:
collecting, using a data collection module, a plurality of physiological scan interpretations for the EEG;
transmitting at least some of the plurality of physiological scan interpretations to an EEG interpretation reliability score module;
automatically calculating, using the reliability score module, an intra-rater reliability score and an inter-rater reliability score for the physiological scan based on the plurality of physiological scan interpretations;
over an extended period of time:
collecting, using the data collection module, additional physiological scan interpretations for the physiological scan;
transmitting the additional physiological scan interpretations to the reliability score module;
automatically recurrently recalculating, using the reliability score module, the intra-rater reliability score and the inter-rater reliability score using the additional physiological scan interpretations; and
providing, to a user interface, the recalculated intra-rater reliability score and the recalculated inter-rater reliability score to provide on-going monitoring,
wherein at least a first physiological scan interpretation and a second physiological scan interpretation are received from different raters,
wherein the additional physiological scan interpretation in the plurality of physiological scan interpretations includes at least one diagnosis, wherein the at least one diagnosis is one of seizure, status epilepticus, normal, burst suppression, epileptiform, slowing, triphasic waves, technically limited, or technically inadequate, and
wherein the additional physiological scan interpretation in the plurality of physiological scan interpretations includes supplemental interpretation information.
